(19) United States
(12) Patent Application Publication
(10) Pub. No.: US 2012/0269720 A1
(43) Pub. Date: Oct. 25, 2012

(54) DETECTION OF HEPATIC INSULIN RESISTANCE

(75) Inventor: David L. Silver, Singapore (SG)

(73) Assignee: Albert Einstein College of Medicine of Yeshiva University, Bronx, NY (US)

(21) Appl. No.: 13/388,373

(22) PCT Filed: Jun. 16, 2010
(86) PCT No.: PCT/US10/01726

§ 371 Date: Jul. 9, 2012

Related U.S. Application Data
(60) Provisional application No. 61/274,172, filed on Aug. 13, 2009.

(51) Int. Cl. A61K 49/00; A61K 51/00; C07H 21/04; C07K 2/00; C07K 16/18; G01N 21/17; C12Q 1/68

(52) U.S. Cl. 424/1.11; 435/7.1; 424/9.1; 435/6.12; 530/300; 536/23.1; 530/389.8; 530/391.3; 536/24.31; 435/6.11

(57) ABSTRACT

Disclosed are methods for determining whether a subject may have hepatic insulin resistance, for diagnosing hepatic insulin resistance, and for assessing the prognosis of a subject with hepatic insulin resistance, comprising determining the level of expression of major facilitator superfamily domain 2 protein (Mfsd2) in a subject's liver, where overexpression of Mfsd2 is indicative of hepatic insulin resistance.

### CROSS-REFERENCE TO RELATED APPLICATION

This application claims priority of U.S. Provisional Patent Application No. 61/274,172, filed on Aug. 13, 2009, the content of which is incorporated by reference.

### BACKGROUND OF THE INVENTION

Various publications are referred to in parentheses throughout this application. Full citations for these references may be found at the end of the specification immediately preceding the claims. The disclosures of these publications are hereby incorporated by reference in their entireties into the subject application to more fully describe the art to which the subject application pertains.

It is reported by the Centers for Disease Control that approximately 7.8% of the US population has type 2 diabetes, which is a leading cause of death and disability and costs an estimated $174 billion each year in health care-related costs. The incidence of type 2 diabetes worldwide is estimated to rapidly increase from 171 million in the next two decades to 366 million as a direct result of the worldwide obesity epidemic (Wild et al., 2004). More troubling is the sharp rise in childhood obesity and type 2 diabetes (Mokdad et al., 2003). Hepatic insulin resistance is an early precondition that leads to type 2 diabetes (Savage et al., 2007). A major unmet challenge is to develop a biomarker that can detect hepatic insulin resistance years to decades in advance of the onset of type 2 diabetes in order to initiate preventative treatments such as lifestyle interventions. Such a clinical diagnostic tool could result in improved quality of life and save the US billions in health care costs and loss in productivity due to diabetes.

Current diagnostics for hepatic insulin resistance are limited. Enhanced hepatic gluconeogenesis is arguably the primary cause of loss of glycemic control in humans (Petersen et al., 2006; Shulman 1999). Hepatic insulin resistance precedes loss of glycemic control by many years (Petersen et al., 2006; Shulman 1999) making it plausible that having a biomarker that can robustly report hepatic insulin resistance may lead to early clinical intervention and inform on treatment modalities.

The hyperinsulinemic euglycemic clamp is currently the only means to measure hepatic insulin resistance. However, this approach is time consuming, difficult to interpret and expensive and is not a clinical diagnostic tool but only used as an experimental tool. Homostasis model assessment of insulin resistance (HOMA-IR) index is a mathematical model that uses the product of insulin and glucose concentration to predict whole body insulin resistance, and does not detect hepatic insulin resistance; in fact at the point at which a HOMA-IR is indicating insulin resistance, the patient has poor glycemic control and is well on the way to type 2 diabetes (Wallace et al., 2004). Potential surrogate markers for insulin resistance have been proposed and experimentally tested such as C-reactive Protein, IL-6, liver transaminases (e.g. GGT) and RBP4. All have shown weak correlations with insulin resistance, and none are direct readouts of hepatic insulin resistance (Reinehr et al., 2009; Greenfield et al., 2006; Pfutzner et al., 2009). In summary, there are no current clinical diagnostic tools for measuring hepatic insulin resistance.

Due to the high incidence of type 2 diabetes, the worldwide obesity epidemic that is estimated to sharply increase the incidence of type 2 diabetes, and the high cost of diabetes, there is a need for a biomarker that can detect hepatic insulin resistance years before the onset of type 2 diabetes, which would allow the implementation of preventative treatment.

### SUMMARY OF THE INVENTION

The invention provides a method for determining whether a subject may have hepatic insulin resistance comprising testing the subject's liver and/or tissue sample for expression of major facilitator superfamily domain 2 protein (Mfsd2), wherein overexpression of Mfsd2 is indicative of hepatic insulin resistance and lack of overexpression of Mfsd2 is indicative that the subject does not have hepatic insulin resistance.

The invention also provides a method for diagnosing hepatic insulin resistance in a subject comprising imaging the subject's liver and/or tissue sample, wherein overexpression of Mfsd2 indicates increased hepatic insulin resistance.

The invention further provides a method for assessing the prognosis of a subject with hepatic insulin resistance, the method comprising imaging the subject's liver and/or tissue sample for expression of Mfsd2, wherein the subject's prognosis increases with a decrease in expression of Mfsd2.

The invention provides kits for detecting the expression of Mfsd2, where the kit comprises an antibody, aptamer, peptide or small molecule that specifically binds to Mfsd2.

### BRIEF DESCRIPTION OF THE DRAWINGS

FIG. 1. A mechanism for lipid-induced hepatic insulin resistance. In the insulin-sensitive liver AKT2 normally phosphorylates Foxo1 inhibiting its nuclear import, and decreasing gluconeogenic gene expression. Increased hepatic pools of diacylglycerol (DAG) derived from fatty acid uptake or de novo fatty acid biosynthesis mediated by fatty acid Synthase (FAS) activates PKC-ε which binds to and inactivates the insulin receptor kinase resulting in reduced insulin signaling to AKT2. AKT2 normally phosphorylates Foxo1 inhibiting its nuclear import, but reduced activation of AKT2 results in increased nuclear levels of Foxo1. Nuclear Foxo1 induces gluconeogenic gene expression such as phosphoenolpyruvate carboxykinase and glucose-6-phosphatase resulting in increased hepatic glucose production and hyperglycemia.

FIG. 2. Fasting-induced expression of Mfsd2. Northern blot analysis indicated that Mfsd2 mRNA levels in livers of 4 fasted mice (12 hrs) were increased relative to 4 fed mice. All mice were 12 week old wild-type males. The ethidium bromide stained gel is show to indicate similar loading.

FIG. 3. Tissue distribution of mouse Mfsd2. Northern blot analysis indicated that Mfsd2 mRNA is expressed highest in liver with lower levels in stomach, kidney, small intestine, testis and ovaries. Tissues were collected from fasted (12 hrs) mice. The ethidium bromide stained gel is shown to indicate loading.

FIG. 4. Membrane topology of human Mfsd2. TMHMM v. 2.0 transmembrane domain prediction (hidden Markov model) of human Mfsd2 indicates 10 or 12 transmembrane domains with both N- an C-termini cystolic. Numbering in top row indicates extracellular loops. Amino acid sequence numbering shown on the ordinate.

FIG. 5. Human Mfsd2 expression is induced by Foxo1. The human hepatocyte cell line HepG2 were transduced with either control adenovirus (adEmpty) or a dominant active Foxo1 adenovirus. Foxo1DA activated expression of human Mfsd2. Data were generated by Taqman qRT-PCR and represented as normalized mean expression relative to adEmpty ±SD. *P<0.002.

FIG. 6A-6B. Increased expression of Mfsd2 in diabetic mouse models. A. Western blot analysis indicated that Mfsd2 is increased in livers of mice fed a high fat diabetogenic diet, HFD (59% calories from fat), for 12 weeks and insulin resistant and diabetic ob/ob mice compared to normal diet fed mice (chow). Data from 2 chow fed, and 3 HJFD, and 2 ob/ob mice are shown. Calnexin served as a loading control. B. Plasma glucose levels were increased in mice fed HFD and in ob/ob compared to control chow fed mice; n=4 per group. Data are represented as mean ±SD. *P<0.00001, chow vs. HFD or ob/ob.

FIG. 7. Mfsd2 sequence alignment. ClustalW alignment analysis of human Mfsd2 (SEQ ID NO:1) and mouse Mfsd2 (SEQ ID NO:2) indicates that these proteins are 84% identical. CON=Consensus.

### DETAILED DESCRIPTION OF THE INVENTION

The invention provides a method for determining whether a subject may have hepatic insulin resistance comprising determining the level of expression of Mfsd2 in the subject's liver or sample thereof, wherein overexpression of Mfsd2 is indicative that the subject has hepatic insulin resistance or wherein no overexpression of Mfsd2 is indicative that the subject does not have hepatic insulin resistance.

The invention also provides a method for diagnosing hepatic insulin resistance in a subject comprising imaging the subject's liver and/or tissue sample, wherein overexpression of Mfsd2 indicates increased hepatic insulin resistance or wherein no overexpression of Mfsd2 may indicate a lack of hepatic insulin resistance.

The invention further provides a method for assessing the prognosis of a subject with hepatic insulin resistance, the method comprising imaging the subject's liver and/or tissue sample for expression of Mfsd2, wherein the subject's prognosis increases with a decrease in expression of Mfsd2 or wherein the subject's prognosis decreases with an increase in expression of Mfsd2.

The subject's liver can be imaged using an agent that specifically binds to Mfsd2. Mfsd2 is expressed on the basolateral membrane of the liver. The agent can, for example, be an antibody, an antibody fragment, an aptamer, a peptide, or a small molecule. The agent can be labeled with a detectable marker. The subject's liver can be imaged using positron emission tomography (PET) or single photon emission computer tomography (SPECT) analysis. Alternatively, a sample of the subject's liver can be assayed using immunological techniques, for example, the Western blot. A sample of the subject's liver can be assayed for the presence of Mfsd2 mRNA by, for example, Real-time quantitative PCR.

The invention provides for a kit to detect expression of Mfsd2, the kit comprising an antibody, an antibody fragment, an aptamer, peptide, or small molecule that specifically binds to Mfsd2. The antibody, antibody fragment, aptamer, peptide or small molecule may be labeled with a detectible marker. The kit may additionally comprise instructions for use comprising the steps of, for example, (a) administering the antibody, antibody fragment, aptamer, peptide or small molecule to a subject; and (b) imaging the subject's liver.

The invention further provides for a kit for detecting the presence of Mfsd2 mRNA, the kit comprising a probe that specifically binds to Mfsd2 mRNA. The probe may be labeled with a detectable marker. The kit may additionally comprise instructions for use comprising the steps of, for example, (a) taking a sample of the subject's hepatic tissue; (b) running a Northern Blot.

As used herein, overexpression of Mfsd2 means overexpression relative to the level in normal hepatic tissue under the same physiological conditions. Under fed conditions, the expression of Mfsd2 in normal hepatic tissue nears undetectable levels. Overexpression is therefore the expression of any detectable level of Mfsd2 in hepatic tissue under fed conditions. This obviates the need for a control in determining overexpression. The higher a subject's expression of Mfsd2 in hepatic tissue in the fed state, the higher the risk of progressing to type-2 diabetes. By determining the overexpression of Mfsd2 in hepatic tissue in a fed state of a subject during the course of treatment for hepatic insulin resistance, efficacy of the treatment can be monitored. If a subject's overexpression of Mfsd2 in hepatic tissue under fed conditions does not decrease, or increases, over time or during the course of treatment, the subject faces a higher risk of progressing to type-2 diabetes.

The expression of Mfsd2 may be detected in vitro or in vivo by detection methods readily determined from the known art, including, without limitation, imaging techniques such as PET and SPECT. The expression of Mfsd2 may be detected in a sample of tissue by detection methods readily determined from the known art, including, without limitation, immunological techniques such as the Western blot or imaging techniques such as PET and SPECT.

The overexpression of Mfsd2 may additionally be detected in a sample of tissue by an analysis of Mfsd2 mRNA levels in a sample of liver tissue from a subject. The presence of Mfsd2 mRNA in the liver tissue from a fed subject indicates overexpression of Mfsd2 in hepatic tissue in the fed state. The presence of Mfsd2 mRNA in liver tissue can be detected by methods readily determined from the known art, including, without limitation, techniques such as Real-time quantitative PCR.

The human protein sequence for Mfsd2 is:

(SEQ ID NO: 1)
makgegaesg saagllptsi lqsterpaqv kkepkkkkqq lsvcnklcya lggapyqvtg calgfflqiy lldvaqvgpf
sasiilfvgr awdaitdplv glciskspwt clgrlmpwii fstplaviay fliwfvpdfp hgqtywyllf yclfetmvtc
fhvpysaltm fisteqterd satayrmtve vlgtvlgtai qgqivgqadt pcfqdlnsst vasqsanhth gttshretqk
ayllaagviv ciyiicavil ilgvreqrep yeaqqsepia yfrglrlvms hgpyiklitg flftslafml vegnfvlfct
ytlgfrnefq nlllaimlsa tltipiwqwf ltrfgkktav yvgissavpf lilvalmesn liityavava agisvaaafl
lpwsmlpdvi ddfhlkqphf hgtepiffsf yvfftkfasg vslgistlsl dfagyqtrgc sqpervkftl nmlvtmapiv
lillglllfk mypideerrr qnkkalqalr deasssgcse tdstelasil.

The mouse protein sequence for Mfsd2 is:

(SEQ ID NO: 2)
makgegaesg saagllptsi lqaserpvqv kkepkkkqql sicnklcyav ggapyqltgc algfflhiyl ldvakveplp
asiilfvgra wdaftdplvg fcisksswtr lgrlmpwiif stplaiiayf liwfvpdfps gtesshgflw yllfyclfet
lvtcfhvpys altmfisteq serdsatayr mtvevlgtvi gtaiqgqivg qakapclqdq ngsvvvseva nrtqstaslk
dtqnayllaa giiasiyvlc afililgvre qrelyesqqa esmpffqglr lvmghgpyvk liagflftsl afmlvegnfa
lfctytldfr nefqnlllai mlsatftipi wqwfltrfgk ktavyigiss avpflilval mernlivtyv vavaagvsva
aafllpwsml pdviddfhlk hphspgtepi ffsfyvfftk fasgvslgvs tlsldfanyq rqgcsqpeqv kftlkmlvtm
apiilillgl llfklypide elrrqnkkal qalreeasss gcsdtdstel asil.

The expression of Mfsd2 can be detected by using an agent that specifically binds to a portion of Mfsd2. The agent can be, for example, an antibody, an aptamer, a peptide, or a small molecule. As used herein, the term “antibody” encompasses whole antibodies and fragments of whole antibodies wherein the fragments specifically bind to Mfsd2. The term antibody is further mean to encompass polyclonal antibodies and monoclonal antibodies. Antibodies may be produced by techniques well known to those skilled in the art. The antibody can be a human antibody or a non-human antibody such as goat antibody or a mouse antibody. Antibodies can be “humanized” using standard recombinant DNA techniques.

Aptamers are single stranded oligonucleotides or olignucleotide analogs that bind to a particular target molecule, such as a protein. Thus, aptamers are the oligonucleotide analogy to antibodies. However, aptamers are smaller than antibodies. Their binding is highly dependent on the secondary structure formed by the aptamer oligonucleotide. Both RNA and single stranded DNA (or analog) aptamers can be used. Aptamers that bind to virtually any particular target can be selected using an iterative process called SELEX, which stands for Systematic Evolution of Ligands by Exponential enrichment.

The agent is administered to the subject prior to imaging the subject's liver. Alternatively, the agent is administered to a sample of the subject's hepatic tissue prior to imaging.

The agent that specifically binds to Mfsd2 may be labeled with a detectable marker. Labeling may be accomplished using one of a variety of labeling techniques, including radioactive labels, peroxidase, and/or chemiluminescent known in the art. The detectable market may be, for example, a radioactive marker, including, for example, a radioisotope or radionuclide. Radioactivity emitted can be detected by techniques well known in the art including, for example, PET and/or SPECT.

### EXPERIMENTAL DETAILS

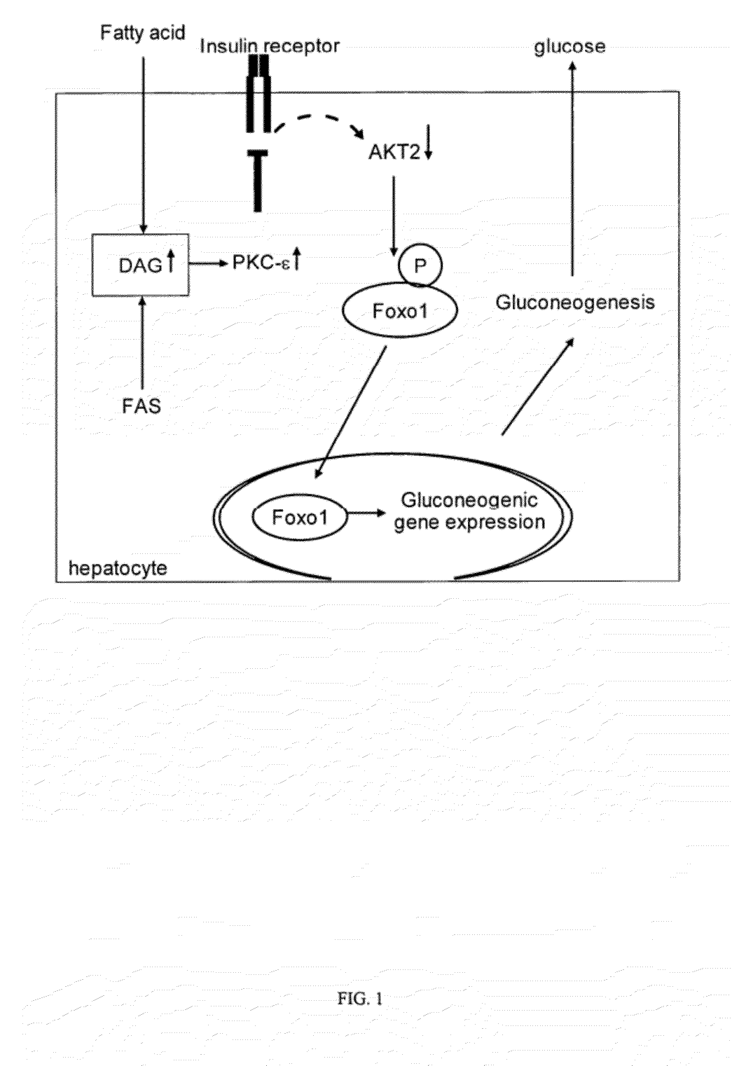

Introduction. There are currently no reliable methodologies for detecting hepatic insulin resistance. Mfsd2 is identified as a liver-enriched cell surface protein induced by Foxo1, the master transcriptional regulator of hepatic glucogenesis and a direct downstream target of insulin signal transduction in the liver. Dysregulation of Foxo1 in rodents and humans as a result of increased lipid deposition in the liver has been shown to be directly correlated with hepatic insulin resistance (Valenti et al., 2008; Puigserver et al., 2003; Matsumoto et al., 2006; Nakae et al., 2001; Frescas et al., 2005; Qu et al., 2006; Matsumoto et al., 2007; Samuel et al., 2006; Altomonte et al., 2003) (FIG. 1). Therefore, the a cell surface, liver-specific protein encoded by a Foxo1-regulated gene could be used to develop imaging reagents to measure hepatic insulin resistance in humans.

Thus far, Foxo1 targets that are liver-specific are intracellular proteins such as PEPCK, pyruvate carboxylase, and glucose-6-phsophatase making them poor targets for in vivo imaging in humans. Major facilitator superfamily domain 2 protein (Mfsd2) is identified as a liver-enriched cell surface protein induced by Foxo 1. Mfsd2 can be used as a robust and sensitive readout for hepatic insulin resistance in humans. By using Mfsd2 as a target for PET imaging of hepatic insulin resistance, hepatic insulin resistance can be determined, diagnosed, and treated many years prior to the onset of type II diabetes. Additionally, the probability of a subject becoming a type II diabetic can be prognosticated.

During an early fast, the liver is the primary organ for glucose production to maintain energy balance mainly by feeding the brain. Hepatic glucose production results from the breakdown of glycogen during the early stages of a fast, but glycogen stores are rapidly depleted within several hours. During later stages of a fast, hepatic glucose production is driven by gluconeogenesis (glucose biosynthesis) that utilizes glycerol, amino acids, and lactate to produce glucose. The rate limiting enzymes in hepatic gluconeogenesis (PC, PEPCK, Glucose-6-phosphatase) are tightly regulated at the transcriptional level primarily through the activity of Foxo1 transcription factor in response to lowering plasma insulin and increased glucagon. Foxo1 is a member of the forked head-winged X factor that is a direct substrate of Akt/PKB phosphorylation, such that in the fed state insulin signaling induces the activation of Akt and phosphorylation of Foxo1 and nuclear exclusion (Matsumoto et al., 2005) (FIG. 1). In the fasted state with low levels of insulin, non-phosphorylated Foxo1 localizes to the nucleus and activates genes important in gluconeogenesis. Therefore, nuclear levels of Foxo1 directly indicate the functioning of insulin signal transduction. Foxo1's role as a master regulator of hepatic glucose production and as an indicator of hepatic insulin resistance is evidenced by the fact that Adenoviral overexpression of dominant active Foxo1 (constitutively localized in the nucleus) in mice results in enhanced gluconeogenesis, hyperglycemia and impaired insulin suppression of gluconeogenesis (Matsumoto et al., 2006; Qu et al., 2006). The converse is found when Foxo1 dominant negative adenovirus is expressed in mice or genetic knockdown experiments are performed, such that Foxo1 defective or deficient mice have decreased gluconeogenesis and hypoglycemia (Qu et al., 2006; Matsumoto et al., 2007; Samuel et al., 2006; Altomonte et al., 2003). Diabetic mice (db/db) expressing the dominant negative Foxo1 showed markedly improved fasting glycemia, correlating with decreased gluconeogenic gene expression and hepatic glucose output (Qu et al., 2006). Indeed, diabetic mice (db/db or liver insulin receptor knockout mice) have increased nuclear Foxo1 and increased gluconeogenic gene expression (Qu et al., 2006). The relevance of nuclear Foxo1 to human hepatic insulin resistance was recently verified in humans having hepatic steatosis or nonalcoholic steatohepititis (NASH). Nuclear Foxo1 levels were increased in patients with hepatic steatosis or NASH compared to normal livers and highly correlated with insulin resistance (Valenti et al., 2008). Taken together, Foxo1 acts as the primary regulator of gluconeogenesis and it's nuclear activity or levels are directly correlated with hepatic insulin resistance.

Results and Discussion. Mfsd2 is nearly undetectable in livers from fed, normal glycemic mice, but highly induced in livers in two different conditions: physiologically during a fast, and pathophysiologically in insulin resistant states. Moreover, the regulation of Mfsd2 is mediated by the insulin-sensitive transcription factor Foxo1. Additionally, Mfsd2 is localized to the basolateral membrane of liver making it easily accessible to antibodies or small molecules in blood for PET imaging.

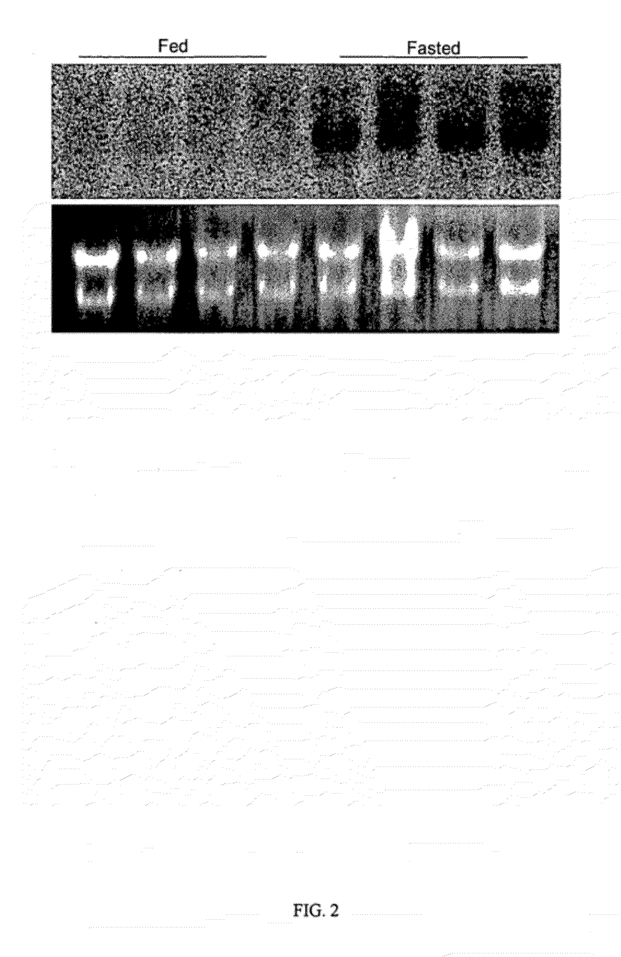

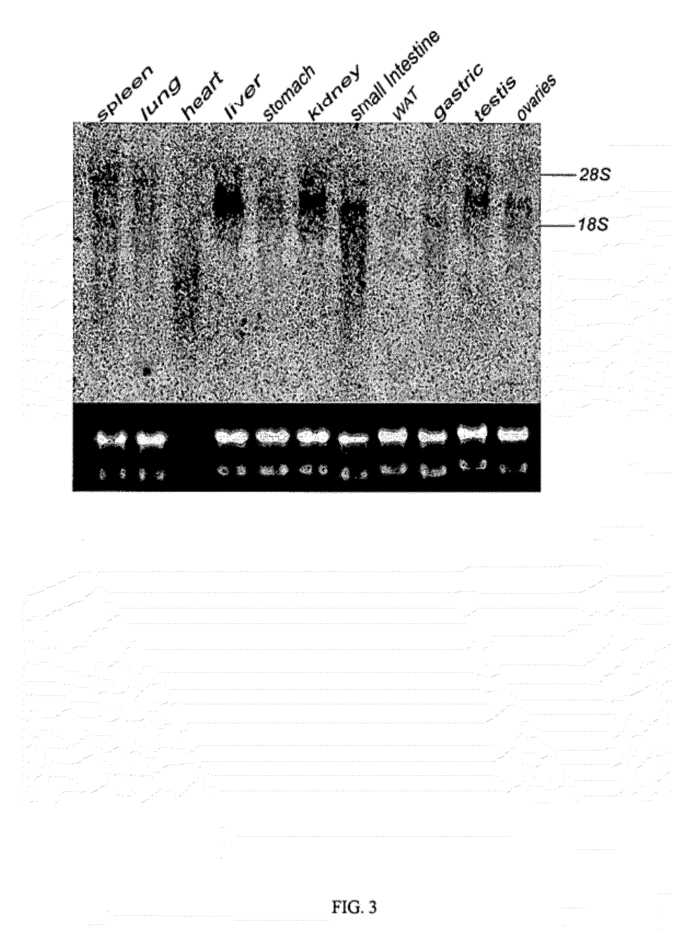

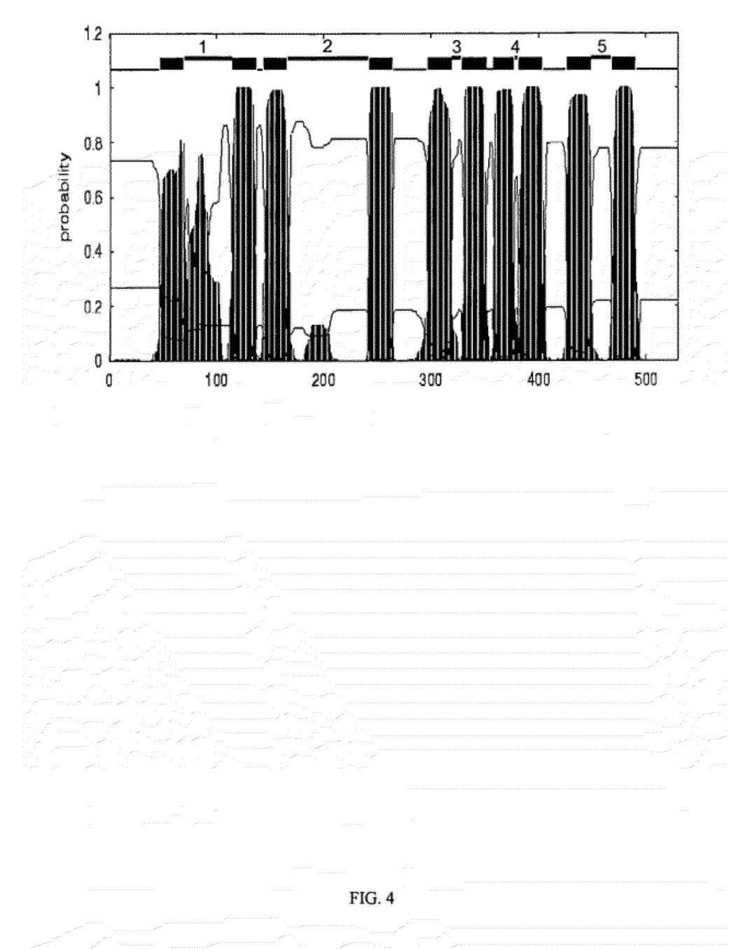

Mfsd2 was identified as a fasting-induced transcript, indicative of a Foxo1 regulated gene, using Affymetrix microarrays and confirmed by Northern blot analysis of mouse liver (FIG. 2). Mfsd2 is undetectable in livers from fed, normal glycemic mice (FIG. 2). Mfsd2 is expressed at low levels in kidney, stomach, small intestine, testis and ovaries, with highest expression in liver of fasted mice (FIG. 3). Mouse and human Mfsd2 are predicted to have 10 to 12 transmembrane domains (FIG. 4) and are highly homologous to each other (84% identical). Mfsd2 is part of a super family of hexose transporters with homologues conserved back to E. coli.

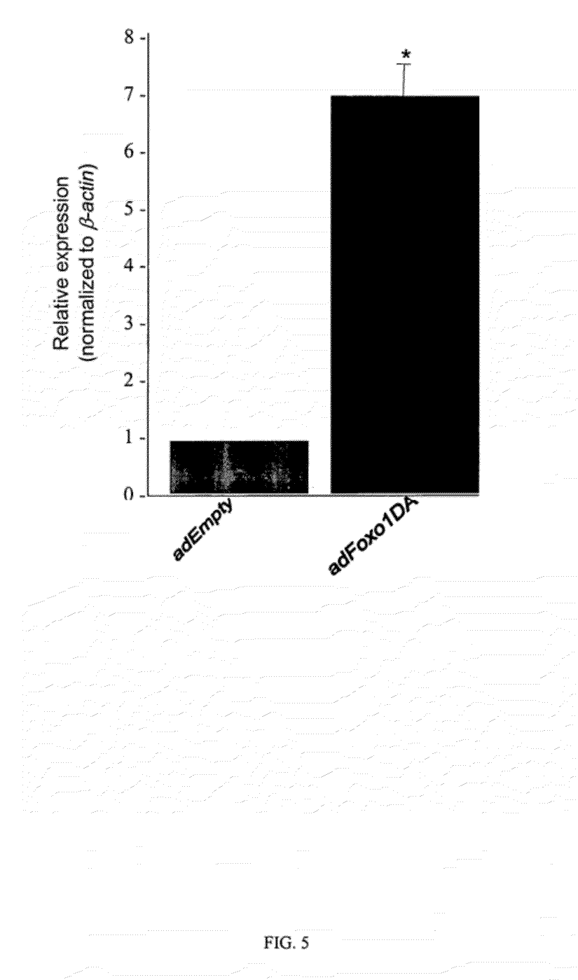

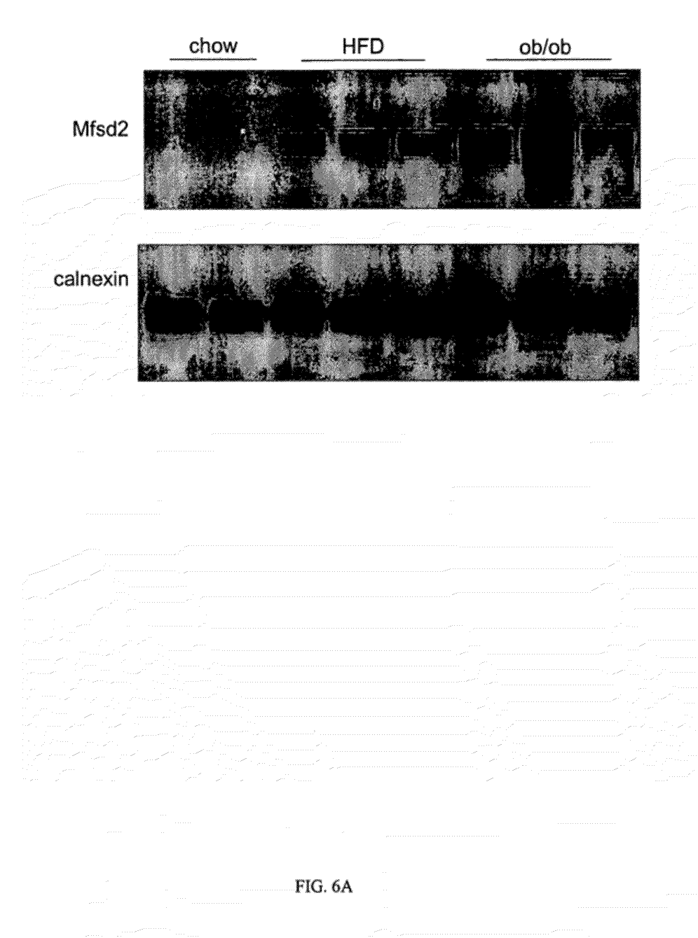

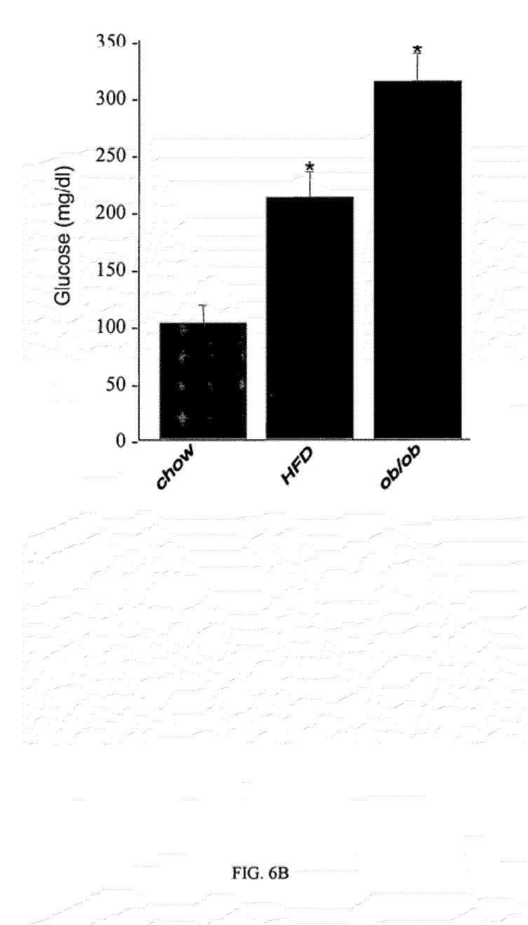

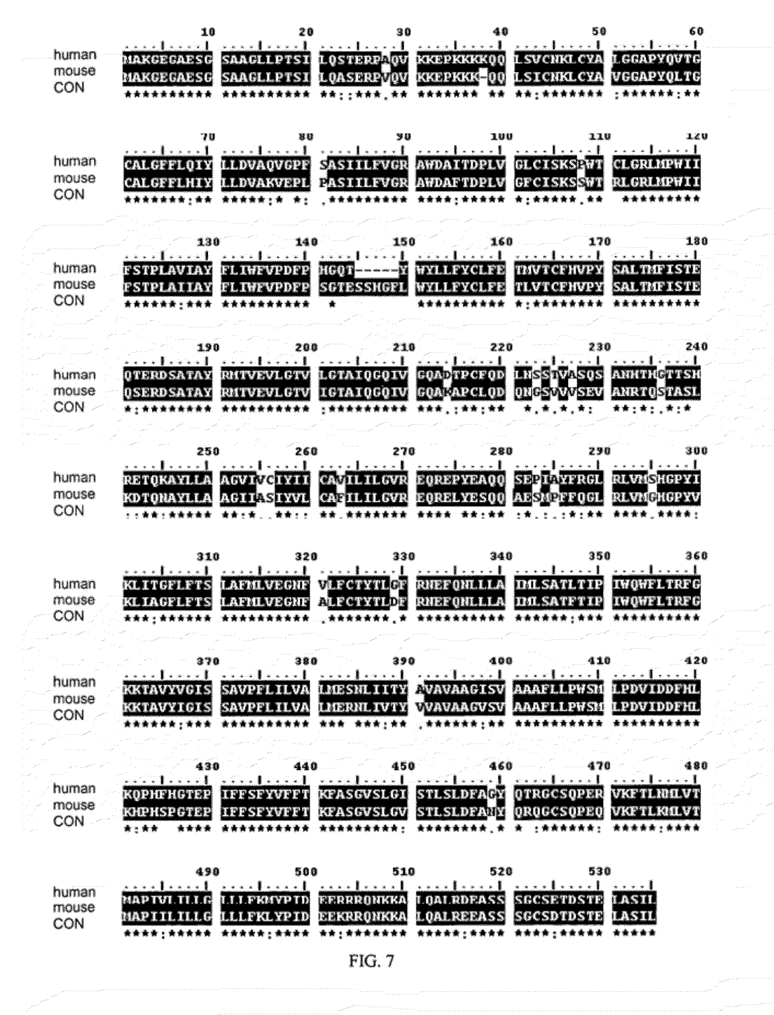

Mfsd2 is fasting-induced indicated potential regulation by Foxo1. Overexpression of a dominant active Foxo1 in a human derived hepatocyte cell line resulted in a marked induction of human Mfsd2 expression (FIG. 5). It is well known that hepatic insulin resistance leads to inappropriate nuclear translocation of Foxo1 (FIG. 1). Two mouse models having hepatic insulin resistance, high fat diet fed mice and leptin deficient ob/ob mice showed marked increases in hepatic Mfsd2 protein under fed conditions when Mfsd2 is absent in wild-type normal glycemic controls (FIG. 6), strongly indicating that Mfsd2 is a biomarker for hepatic insulin resistance, corroborating the finding that it is a Foxo1-regulated gene.

For Mfsd2 to be used as a biomarker imaging tool for clinically diagnosing hepatic insulin resistance, Mfsd2 must be expressed on the sinusoidal (blood-facing) side of hepatocytes, thereby being accessible to positron emission tomography labeled molecules injected intravenously in subjects. Expression of wild-type Mfsd2 (not epitope tagged) in HEK293 cells in culture resulted in plasma membrane expression. Importantly, Mfsd2 was expressed on the sinusoidal (blood-facing) membrane of hepatoctyes in mouse livers.

### REFERENCES

Altomonte, J., Richter, A., Harbaran, S., Suriawinata, J., Nakae, J., Thung, S. N., Meseck, M., Accili, D., and Dong, H. 2003. Inhibition of Foxo1 function is associated with improved fasting glycemia in diabetic mice. Am J Physiol Endocrinol Metab 285:E718-728.

Esnault, C., Priet, S., Ribet, D., Vernochet, C., Bruls, T., Lavialle, C., Weissenbach, J., and Heidmann, T. 2008. A placenta-specific receptor for the fusogenic, endogenous retrovirus-derived, human syncytin-2. Proc Natl Acad Sci U S A 105:17532-17537.

Frescas, D., Valenti, L., and Accili, D. 2005. Nuclear trapping of the forkhead transcription factor FoxO1 via Sirt-dependent deacetylation promotes expression of glucogenetic genes. J Biol Chem 280:20589-20595.

Greenfield, J. R., and Campbell, L. V. 2006. Relationship between inflammation, insulin resistance and type 2 diabetes: ‘cause or effect’? Curr Diabetes Rev 2:195-211.

Matsumoto, M., and Accili, D. 2005. All roads lead to FoxO. Cell Metab 1:215-216.

Matsumoto M., Han S., Kitamura T., Accili, D. 2006. Dual role of transcription factor FoxO1 in controlling hepatic insulin sensitivity and lipid metabolism. J Clin Invest 116:2464-72.

Matsumoto, M., Pocai, A., Rossetti, L., Depinho, R. A., and Accili, D. 2007. Impaired regulation of hepatic glucose production in mice lacking the forkhead transcription factor Foxo1 in liver. Cell Metab 6:208-216.

Mokdad A. H., Ford E. S., Bowman B. A., Dietz W. H., Vinicor F., Bales V. S., Marks, J. S. 2003. Prevalence of obesity, diabetes, and obesity-related health risk factors, 2001. JAMA 289:76-79.

Nakae, J., Kitamura, T., Silver, D. L., and Accili, D. 2001. The forkhead transcription factor Foxo1 (Fkhr) confers insulin sensitivity onto glucose-6-phosphatase expression. J Clin Invest 108:1359-1367.

Petersen, K. F., Shulman, G. I. 2006. Etiology of insulin resistance. Am J Med 119:S10-16.

Pfutzner, A., Schondorf, T., Hanefeld, M., Lubben, G., Kann, P. H., Karagiannis, E., Wilhelm, B., and Forst, T. 2009. Changes in insulin resistance and cardiovascular risk induced by PPARgamma activation have no impact on RBP4 plasma concentrations in nondiabetic patients. Horm Metab Res 41:202-206.

Puigserver, P., Rhee, J., Donovan, J., Walkey, C. J., Yoon, J. C., Oriente, F., Kitamura, Y., Altomonte, J., Dong, H., Accili, D., et al. 2003. Insulin-regulated hepatic gluconeogenesis through FOXO1-PGC-lalpha interaction. Nature 423:550-555.

Qu, S., Altomonte, J., Perdomo, G., He, J., Fan, Y., Kamagate, A., Meseck, M., and Dong, H. H. 2006. Aberrant Forkhead box O1 function is associated with impaired hepatic metabolism. Endocrinology 147:5641-5652.

Reinehr, T., Schmidt, C., de Sousa, G., and Andler, W. 2009. Association between leptin and transaminases: 1-year follow-up study in 180 overweight children. Metabolism 58:497-503.

Samuel V. T., Cho, C. S., Phillips T. G., Romanelli A. J., Geisler J. G., Bhanot S., McKay R., Monia B., Shutter J. R., Lindberg R. A., et al. 2006. Targeting foxo1 in mice using antisense oligonucleotide improves hepatic and peripheral insulin action. Diabetes 55:2042-50.

Savage, D. B., Petersen, K. F., and Shulman, G. I. 2007. Disordered lipid metabolism and the pathogenesis of insulin resistance. Physiol Rev 87:507-520.

Shulman, G. I. 1999. Cellular mechanisms of insulin resistance in humans. Am J Cardiol 84:3J-10J.

Valenti, L., Rametta, R., Dongiovanni, P., Maggioni, M., Fracanzani, A. L., Zappa, M., Lattuada, E., Roviaro, G., and Fargion, S. 2008. Increased expression and activity of the transcription factor FOXO1 in nonalcoholic steatohepatitis. Diabetes 57:1355-1362.

Wallace, T. M., Levy, J. C., and Matthews, D. R. 2004. Use and abuse of HOMA modeling. Diabetes Care 27:1487-1495.

Wild, S., Roglic, G., Green, A., Sicree, R., and King, H. 2004. Global prevalence of diabetes: estimates for the year 2000 and projections for 2030. Diabetes Care 27:1047-1053.

1. A method for determining whether a subject may have hepatic insulin resistance comprising determining the level of expression of major facilitator superfamily domain 2 protein (Mfsd2) in the subject's liver or sample thereof, wherein overexpression of Mfsd2 may indicate hepatic insulin resistance or wherein no overexpression of Mfsd2 may indicate a lack of hepatic insulin resistance.

2. A method for diagnosing hepatic insulin resistance in a subject comprising determining the level of expression of major facilitator superfamily domain 2 protein (Mfsd2) in the subject's liver or sample thereof, wherein overexpression of Mfsd2 indicates increased hepatic insulin resistance or wherein no overexpression of Mfsd2 indicates a lack of hepatic insulin resistance.

3. A method for assessing the prognosis of a subject with hepatic insulin resistance, the method comprising imaging the subject's liver and/or tissue sample for expression of major facilitator superfamily domain 2 protein (Mfsd2), wherein the subject's prognosis increases with a decrease in expression of Mfsd2 or wherein the subject's prognosis decreases with an increase in expression of Mfsd2.

4. The method of claim 1, wherein the subject's liver is imaged using an agent that specifically binds to Mfsd2.

5. The method of claim 4 wherein the Mfsd2 is expressed on the basolateral membrane of the liver.

6. The method of claim 4 wherein the agent is an antibody, an antibody fragment, an aptamer, a peptide, or a small molecule.

7. The method of claim 4 wherein the agent is labeled with a detectable marker.

8. The method of claim 1, wherein the subject's liver is imaged using PET or SPECT analysis.

9. The method of claim 1, 2, or 3 wherein the sample of the subject's liver is assayed using immunological techniques.

10. The method of claim 9 wherein the immunological technique used is Western blot.

11. The method of claim 1, wherein the sample of the subject's liver is assayed for the presence of Mfsd2 mRNA.

12. The method of claim 11 wherein the sample of the subject's liver is assayed for the presence of Mfsd2 mRNA using Real-time quantitative PCR.

13. A kit for detecting expression of Mfsd2, the kit comprising an antibody, an antibody fragment, an aptamer, peptide or small molecule that specifically binds to Mfsd2.

14. The kit of claim 13 wherein the antibody, antibody fragment, aptamer, peptide or small molecule is labeled with a detectable marker.

15. The kit of claim 13 wherein the kit additionally comprises instructions for use.

16. The kit of claim 15 wherein the instructions for use comprise the steps of (a) administering the antibody, antibody fragment, aptamer, peptide or small molecule to a subject; and (b) imaging the subject's liver.

17. A kit for detecting the presence of Mfsd2 mRNA, the kit comprising a probe that specifically binds to Mfsd2 mRNA.

18. The kit of claim 17 wherein the probe is labeled with a detectable marker.

19. The kit of claim 17 wherein the kit additionally comprises instructions for use.

20. The kit of claim 19 wherein the instructions for use comprise the steps of (a) taking a sample of the subject's hepatic tissue; and (b) running a qRT-PCR analysis.


SEQUENCE LISTING (2 sequences)

SEQ ID NO: 1 - protein, 530 aa, Homo sapiens
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 2 - protein, 534 aa, Mus musculus
MAKGEGAESGSAAGLLPTSILQASERPVQVKKEPKKKQQLSICNKLCYAVGGAPYQLTGCALGFFLHIYLLDVAKVEPLPASIILFVGRAWDAFTDPLVGFCISKSSWTRLGRLMPWIIFSTPLAIIAYFLIWFVPDFPSGTESSHGFLWYLLFYCLFETLVTCFHVPYSALTMFISTEQSERDSATAYRMTVEVLGTVIGTAIQGQIVGQAKAPCLQDQNGSVVVSEVANRTQSTASLKDTQNAYLLAAGIIASIYVLCAFILILGVREQRELYESQQAESMPFFQGLRLVMGHGPYVKLIAGFLFTSLAFMLVEGNFALFCTYTLDFRNEFQNLLLAIMLSATFTIPIWQWFLTRFGKKTAVYIGISSAVPFLILVALMERNLIVTYVVAVAAGVSVAAAFLLPWSMLPDVIDDFHLKHPHSPGTEPIFFSFYVFFTKFASGVSLGVSTLSLDFANYQRQGCSQPEQVKFTLKMLVTMAPIILILLGLLLFKLYPIDEEKRRQNKKALQALREEASSSGCSDTDSTELASIL